(19) United States
(12) Patent Application Publication
(10) Pub. No.: US 2003/0139342 A1
(43) Pub. Date: Jul. 24, 2003

(54) Method of inducing vitreous detachment

(76) Inventors: Brooks W. McCuen, Durham, NC (US); Cynthia A. Toth, Chapel Hill, NC (US); David Epstein, Bahama, NC (US); Leonardo deOliveira, Uberlandia (BR); Misako Tatebayashi, Nishinomiya-shi (JP); Carsten H. Meyer, Marburg (DE); Janardan Kumar, Boston, MA (US)

Correspondence Address: NIXON & VANDERHYE, PC, 1100 N GLEBE ROAD, 8TH FLOOR, ARLINGTON, VA, 22201-4714, US

(21) Appl. No.: 10/277,112

(22) Filed: Oct. 22, 2002

Related U.S. Application Data
(63) Continuation of application No. 10/084,653, filed on Feb. 28, 2002, now abandoned.
(60) Provisional application No. 60/271,716, filed on Feb. 28, 2001.

(51) Int. Cl. A61K 38/17

(52) U.S. Cl. 514/12

(57) ABSTRACT

The present invention relates, in general, to a method of treating disorders of the eye, and, in particular, to a method of inducing posterior vitreous detachment.

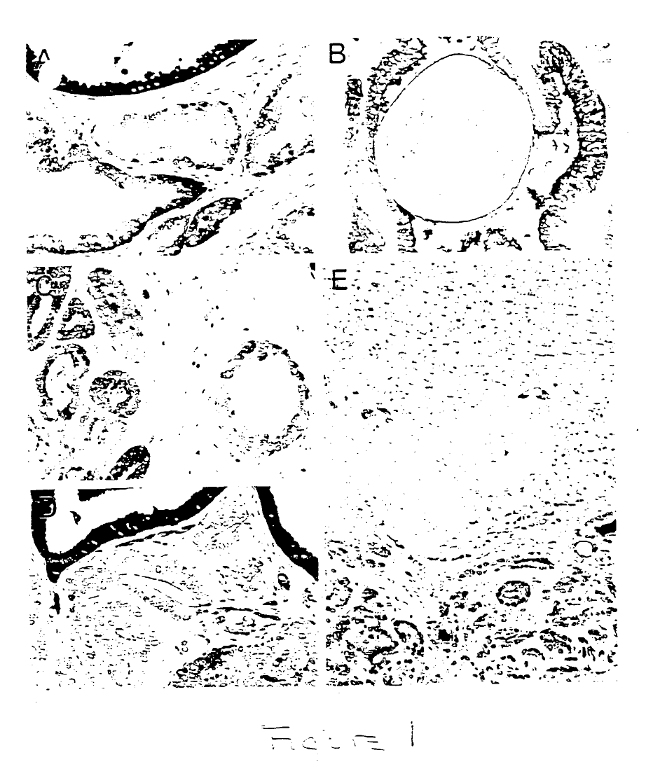

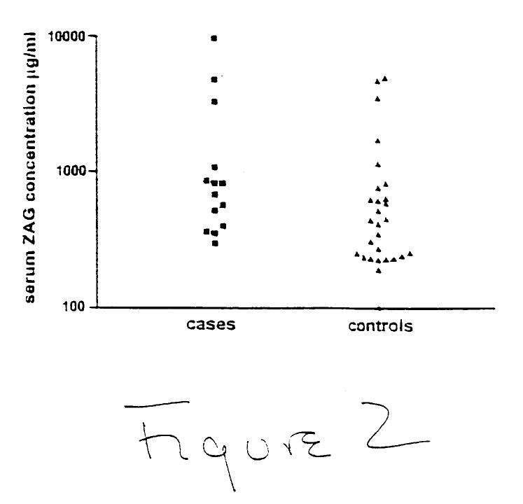

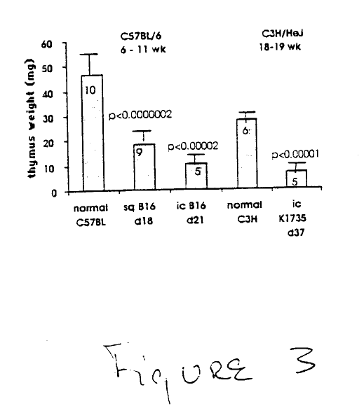

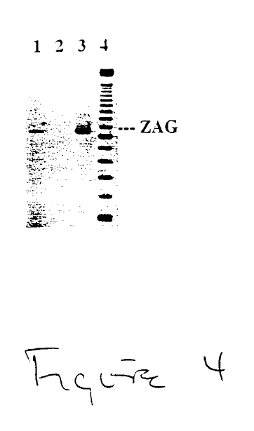

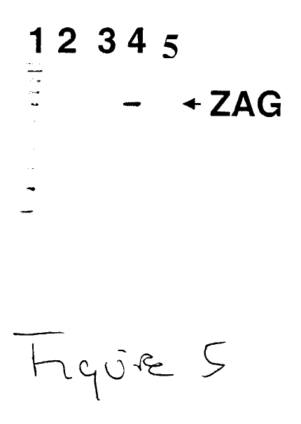

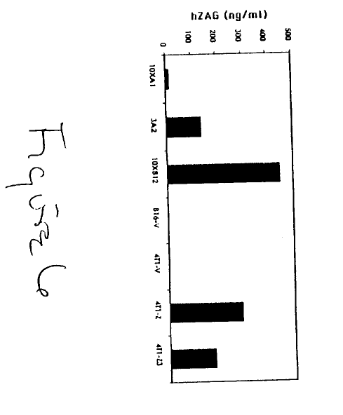

This application claims priority from Provisional Application No. 60/271,716, filed Feb. 28, 2001, the entire content of which is incorporated herein by reference.

### TECHNICAL FIELD

The present invention relates, in general, to a method of treating disorders of the eye, and, in particular, to a method of inducing posterior vitreous detachment.

### BACKGROUND

The vitreous is a gel that occupies approximately 80% of the 5-ml volume of the human eye. Although it is 98% water, its collagen fibrils (˜0.05 to 0.1 mg/ml), hyaluronic acid (˜65 to 250 g/ml) and surface attachment molecules (integrins) define the biomechanics of this material. The posterior hyaloid surface of the vitreous is adherent to the inner retinal surface in the normal human eye, most prominently at the vitreous base, optic disc, and along the major retinal vessels.

The vitreous contains type II collagen as a principal fibrillar collagen as well as collagen types V, IX, and XI. These collagen fibers insert into the retinal internal limiting membrane (ILM). The ILM, which is essentially the basement membrane of retinal Mueller cells, is composed of collagen type IV and is built in 3 layers consisting of the lamina rara interna, lamina densa and lamina externa. The interface is firmly connected by laminin (LN), fibronectin (FN), and glycoconjugates.

The development of an incomplete posterior vitreous detachment (PVD) from the ILM has an impact on many vitreoretinal diseases, e.g., vitreomacular traction syndrome, macular holes, macular edema, diabetic retinopathy, diabetic maculopathy or retinal detachment. Small foci of oersisting abnormally firm attachments of the vitreous can transmit great tractional forces from the vitreous gel to the retina at the attachment site.

Recent studies have explored different enzymes that can be used to promote liquefaction of the vitreous body and separation of the vitreorectinal interface; this process is called pharmacological vitrectomy. Several proteolytic enzymes, e.g., hyaluronidase, dispase, chondrointinase or plasmin, have been injected intravitrealy in experimental and clinical trials to assist this creation of a PVD. However, none of these techniques releases the cleavage of the binding sites between the ILM and the posterior hyaloid completely or without complications. Additionally, the risk of adverse reactions is high as the threshold between effective dosage and toxic dosage is very close.

Various cellular processes such as migration, proliferation, differentiation and activation require cell to cell or cell to extracellular matrix (ECM) contacts. Different ECM-proteins such as FN, LN and a variety of collagens are involved. FN, for example, which is present in the basal lamina, is a major cell adhesive protein to integrin receptors. Integrins are cell-surface receptors for the ECM-proteins that mediate adhesions to the ECM. These receptors consist of two non-covalently linked α and β subunits and are associated with the cell membrane. They recognize an Arg-Gly-Asp-containing peptide sequence, also called RGD-cell-binding sequence, which builds the interaction between integrins and the ECM macromolecules.

The present invention provides a method of disrupting the junction between the vitreous and the retina in the eye using RGD-containing polypeptides, or derivatives or mimetics thereof. The present method, which relieves pathologic vitreous traction, can be used in treating various retinal diseases and supporting the vitreo-retinal surgeon creating an easier and safer PVD during vitrectomy.

### DETAILED DESCRIPTION OF THE INVENTION

The present invention relates to a method of inducing posterior vitreous detachment (PVD) from the retina surface. The method comprises introducing a disintegrin, or derivative or mimetic thereof, into the vitreous cavity under conditions such that PVD is effected.

Disintegrins, or derivatives (including portions of naturally occurring disintegrins) or mimetics thereof, suitable for use in the invention are characterized by their ability to modulate (e.g., interfere with) integrin-ECM interactions. Examples of such compounds include competitive inhibitors of integrin-ECM interactions, such as RGD-containing peptides or amino-peptidase- and/or protease-resistant derivatives thereof (e.g., a D-form of an RGD-containing peptide), which peptides can be synthesized using standard techniques. Specific examples include RGD, GRGDS, GRGDTP, Echistatin (49 amino acids), and Flavoridin (70 amino acids) (a variety of RGD-containing peptides being commercially available (e.g., from Sigma-Aldrich)). Further examples of suitable disintegrins and derivatives thereof, their preparation and properties, are described in numerous publications, as are means of identifying mimetics of disintegrins. See, e.g., Elner et al, Invest. Ophthalmol. Vis. Sci. 37:696-701 (1996); Hershkoviz et al, Invest. Ophthalmol. Vis. Sci. 35:2585-2591 (1994); Kern et al, Eur. J. Biochem. 215:151-159 (1993); Kupper et al, FASEB J. 7:1401-1406 (1993); Williams, Pathol. Biol. (Paris) 40:813-802 (1992); Yamada et al, J. Cell. Physiol. 130:21-28 (1987); Sato et al, J. Cell Biol. 111:1713 (1990); Rucinski et al, Biochim. Biophys. Acta 1054:257 (1990); Gould et al, Proc. Soc. Exp. Biol. Med. 195:168 (1990); Kini et al, Toxicon 28:1387 (1990); Soszka et al, Exp. Cell Res. 196:6 (1991); Calvete et al, Biochemistry 30:5225 (1991); Scarborough et al, J. Biol. Chem. 266:9359 (1991); Adler et al, Biochemistry 31:1031 (1992); Scarborough et al, J. Biol. Chem. 268:1058 (1993); Scarborough et al, J. Biol. Chem. 268:1066 (1993); Omori-Satoh et al, Toxicon 24:1045 (1986); Huang et al, J. Biol. Chem. 262:16157 (1987); Knudsen et al, Exp. Cell Res. 179:42 (1988); Gan et al, J. Biol. Chem. 263:19827 (1988); Huang et al, Biochemistry 28:661 (1989); Garsky et al, Proc. Natl. Acad. Sci. U.S.A. 86:4022 (1989); Shebuski et al, J. Biol. Chem. 264:21550 (1989); Chao et al, Proc. Natl. Acad. Sci. U.S.A. 86:8050 (1989); Musial et al, Circulation 82:261 (1990); Williams et al, Biochim. Biophys. Acta 1039:81 (1990); Savage et al, J. Biol. Chem. 265:11766 (1990); Takeya et al, J. Biol. Chem. 265:16068 (1990); Neeper et al, Nucl. Acids Res. 18:4255 (1990); Seymour et al, J. Biol. Chem. 265:10143 (1990); Shebuski et al, Circulation 82:169 (1990); Dennis et al, Proc. Natl. Acad. Sci. U.S.A. 87:2471 (1990); Mazur et al, Eur. J. Biochem. 2020:1073 (1991); Saudek et al, Eur. J. Biochem. 202:329 (1991); Huang et al, J. Biochem. 109:328 (1991); Adler et al, Science 253:445 (1991); Saudek et al, Biochemistry 30:7369 (1991); Huang et al, Biochim. Biophys. Acta 1074:144 (1991); Huang et al, Biochim. Biophys. Acta 1074:136 (1991); Huang et al, Biochem J. 277:351 (1991); Huang et al, Biochem. Pharmacol. 42:1209 (1991); Yamakawa et al, J. Biochem. 109:667 (1991); Dalvit et al, Eur. J. Biochem. 202:315 (1991); Cooke et al, Eur. J. Biochem. 202:323 (1991); Au et al, Biochem. Biophys. Res. Commun. 181:585 (1991); Chen et al, Biochemistry 30:11625 (1991); Kini et al, Toxicon 30:265 (1992); Hite et al, Biochemistry 31:6203 (1992); Calvete et al, FEBS Letters 309:316 (1992); Sheu et al, Jpn. J. Cancer Res. 83:885 (1992); Paine et al, J. Biol. Chem. 267:22869 (1992); Takeya et al, J. Biol. Chem. 267:14109 (1992); Chang et al, Biochem. Biophys. Res. Commun. 190:242 (1993); Takeya et al, J. Biochem. 113:473 (1993); Adler et al, Biochemistry, 32:282 (1993); Dennis et al, Proteins: Structure. Function and Genetics 15:312 (1993); Hite et al, Arch. Biochem. Biophys. 308:182 (1994).

In addition to the above, anti-integrin antibodies (e.g., monoclonal antibodies), which can be produced using standard techniques, can also be used in the method of the invention.

Test agents can be screened for their suitability for use in the present method by assaying such agents for their ability to inhibit the interaction of integrin with ECM. Assays can be conducted using any of a variety of approaches, including immunohistichemical techniques.

Compounds of the invention can be formulated into compositions suitable for administration to the eye. Compositions comprising the present compounds can be administered, for example, by injection into the mid vitreous of the eye. For topical administration, the compound can be dissolved in a pharmaceutically acceptable carrier substance, e.g., physiological saline. For compounds having limited water solubility, the liquid carrier medium can contain an organic solvent, e.g., 3% methyl cellulose. Methyl cellulose provides, by its high viscosity, increased contact time between the compound and the eye surface, and therefore increased corneal penetration. Corneal penetration can also be increased by administering the compound mixed with an agent that slightly disrupts the corneal membrane, e.g., 0.025% benzalkonium chloride. Administration can comprise periodic application of drops of the compound in solution using an eye dropper, such that an effective amount of the compound is delivered through the cornea. The compounds can also be formulated into gels, ointments or creams that can be applied topically to the tissue surrounding the eye.

Systemic administration of the compounds of the invention is also contemplated, either oral administration or intravenous administration. In the case of oral administration, a suitable composition can be in dosage unit form and is a pill, capsule, tablet or the like. Compositions suitable for intravenous administration are typically formulated as sterile solutions.

Compounds of the invention can also be administered by placing an implant into the eye. Delivery can be effected using a depot or sustained release formulation so that the compound is supplied continuously.

Dosing regimens can be determined readily by one skilled in the art and such regimens will vary with the patient, the compound and the effect sought. Doses to be administered can be, for example, from 0.5-5.0 mg/eye/day.

Whatever the mode of administration, the compositions of the invention include active agent and a pharmaceutically acceptable carrier. The compositions of the invention can also include agents that promote or enhance delivery. The compositions can also include preservatives that prolong shelf life.

The compositions of the invention can be provided in various container means. Compositions to be administered by injection can be provided as solutions in sterile container means.

As indicated above, the development of an incomplete posterior vitreous detachment (PVD) has an impact on many vitreoretinal diseases, e.g., vitremacular traction syndrome, macular holes, macular edema, diabetic retinopathy, diabetic maculopathy or retinal detachment. Small foci of persisting abnormally firm attachments of the vitreous can transmit great tractional forces from the vitreous gel to the retina at the attachment site. During vitrectomy or prior to subretinal surgery, the mechanical separation of the posterior hyaloid has been recommended for two reasons. First, pathological vitreous bonds with cellular structures that may cause traction to the retina. Second, remnants of residual posterior hyaloid can provide the surface for cellular proliferation and contraction complicating the postoperative recovery. The surgical detachment of the cortical vitreous can be difficult and may cause retinal breaks or bleeding. The difficulty during surgery depends on how firmly adhesions between the posterior vitreous hyaloid and the retinal surface namely the ILM are connected. Retinal break formation and bleedings during surgery have important influence on the outcome.

Compounds of the present invention can be used to induce PVD in patients with vitreoretinal diseases and thereby prevent the progression of vitreoretinal disease processes (possibly avoiding the necessity for vitreoretinal surgery). In patients with a pathologic firm attachments, the present compounds can assist the creation of a PVD during vitrectomy and thereby improve the safety and efficacy of this procedure. The creation of PVD in accorance with the present invention can benefit patients that have epiretinal membranes, diabetic retinopathy, macular holes, proliferative vitreoretinopathy and advanced states of retinopathy of prematurity.

Certain aspects of the invention can be described in greater detail in the non-limiting Example that follows.

### EXAMPLE

### Intravitreal Injection of RGD Peptide Facilitates Surgical Creation of a Posterior Vitreous Detachment in Young Pigs

Experimental Details:

To investigate the effectiveness of RGD-peptides in creating a PVD, young living pigs were used as a reliable animal model. The pig eye is closer in structure to the human eye than any other animal, except primates. The retina as well as the vitreo-retinal border have similar structures to those found in humans. In young pigs, the posterior vitreous is completely attached to the retina and a surgical detachment of the vitreous using the mechanical vitreous cutter is extremely difficult or nearly impossible. In prior experiments, this animal model was used successfully to evaluate the effectiveness of dispase to create a PVD.

Prior to vitreous surgery, the RGD-peptide (0.5 or 1.0 mg in 18 eyes) or PBS was injected via the pars plana into the vitreous cavity directly over the optic disc. This was done under indirect observation. Six to twenty-four hours after incubation, the anatomic location of the vitreous to the retinal surface was investigated. Using a conventional vitrectomy, mechanical aspiration was applied to the vitreous close to the optic disc in an attempt to induce a PVD. A determination was made as to whether the posterior vitreous was already detached. If no PVD was visible, lifting of the vitreous was attempted with an aspiration of 25 mmHg.

Intraoperative clincal observation was used, coupled with postoperative ultrasonography, in selected eyes to determine the status of the posterior hyaloid and PVD was classified as follows: Total PVD without evidence of retinal traction; total PVD with evidence of retinal traction; partial PVD; and no PVD.

Results:

The intravitreal injection of 0.5 or 1.0 mg RGD-peptides 6 hours or more prior to vitrectomy facilitated the surgical creation of posterior vitreous separation in pig eyes. The RGD peptide-injected eyes developed a mild vascular exudation about 4 to 6 hours after injection and spontaneously resolved between 2 to 5 days.

All documents cited above are hereby incorporated in their entirety by reference.

What is claimed is:

1. A method of inducing posterior vitreous detachment (PVD) from a retina surface comprising introducing a disintegrin into the vitreous cavity of an eye under conditions such that PVD is effected.

2. A method of inducing posterior vitreous detachment (PVD) from a retina surface comprising introducing an agent into the vitreous cavity of an eye, which agent inhibits integrin-extracellular matrix (ECM) interactions, under conditions such that PVD is effected.

3. The method according to claim 2 wherein said agent is a competitive inhibitor of integrin-ECM interactions.

4. The method according to claim 3 wherein said agent is an RGD-containing peptide, or amino-peptidase- and/or protease-resistant derivative thereof.

5. The method according to claim 4 wherein said agent is a D-form of an RGD-containing peptide.

6. The method according to claim 4 wherein said RGD-containing peptide is RGD, GRGDS, GRGDTP, Echistatin or Flavoridin.

7. The method according to claim 2 wherein said agent is an anti-integrin antibody.

8. The method according to claim 2 wherein said agent is a naturally occurring disintegrin, or derivative, portion or mimetic thereof.

9. A method of screening a test compound for potential as an inducer of posterior vitreous detachment (PVD) from a retina surface comprising contacting integrin, or portion thereof that mediates adhesion to ECM, with an ECM, or RGD-cell binding portion thereof, in the presence and absence of said test compound and assessing the ability of said test compound to inhibit the binding of said integrin, or portion thereof, to said ECM, or portion thereof, wherein a test compounds that inhibits said binding is a potential inducer of said detachment.

10. A method of inducing posterior vitreous detachment (PVD) from a retina surface comprising introducing an inducer identifiable by the method according to claim 9 into the vitreous cavity of an eye under conditions such that PVD is effected.

11. A composition comprising an inducer identifiable by the method according to claim 9 and a carrier, wherein the composition is in a form suitable for administration to the eye.

12. The composition according to claim 11 wherein said composition is in a depot formulation.
